(19) United States
(12) Patent Application Publication
(10) Pub. No.: US 2024/0298949 A1
(43) Pub. Date: Sep. 12, 2024

(54) ANORECTAL TONOMETER FOR NEUROLOGICAL TESTING

(71) Applicant: ARIZONA BOARD OF REGENTS ON BEHALF OF THE UNIVERSITY OF ARIZONA, Tucson, AZ (US)

(72) Inventors: Eniko T. Enikov, Tucson, AZ (US); Rein Anton, Tucson, AZ (US); Gergo Edes, Tucson, AZ (US); Brason R. Holt, Tucson, AZ (US); Tyler B. Fowler, Tucson, AZ (US); Miguel A. Osorio, Tucson, AZ (US)

(21) Appl. No.: 18/667,395

(22) Filed: May 17, 2024

Related U.S. Application Data
(63) Continuation-in-part of application No. PCT/US22/80677, filed on Nov. 30, 2022.
(60) Provisional application No. 63/284,348, filed on Nov. 30, 2021.

(51) Int. Cl. A61B 5/22; A61B 5/00

(52) U.S. Cl. CPC A61B 5/227; A61B 5/742; A61B 2562/0247

(57) ABSTRACT

A rectal tonometer device for quantitatively detecting and measuring a rectal tone of a patient. The device includes a main body including a cavity within and a deformable tip, including a pressure sensor, partially disposed in the cavity and extending from the main body. Deforming the tip may actuate the pressure sensor. The rectal tonometer device further includes a circuit board disposed within the cavity, communicatively coupled to the deformable tip capable of accepting a pressure signal from the pressure sensor and measuring, based on the pressure signal, the rectal tone of the patient.

### CROSS-REFERENCES TO RELATED APPLICATIONS

This application is a continuation-in-part and claims benefit of International Application No. PCT/US2022/080677 filed Nov. 30, 2022, which claims benefit of U.S. Provisional Application No. 63/284,348 filed Nov. 30, 2021, the specification(s) of which is/are incorporated herein in their entirety by reference.

### STATEMENT REGARDING FEDERALLY SPONSORED RESEARCH OR DEVELOPMENT

This invention was made with government support under Grant No. 1446098 awarded by National Science Foundation. The government has certain rights in the invention.

### FIELD OF THE INVENTION

The present invention is directed to an anorectal tonometer capable of quantitative measurement of a rectal tone.

### BACKGROUND OF THE INVENTION

Cauda Equina Syndrome (CES) is a rare condition that affects the Cauda Equina, a bundle of spinal nerve roots running through the lower section of the subarachnoid space which supplies the bladder, bowel, and sexual functions. Despite its particularly low prevalence, CES is considered a major medical emergency internationally and multiple National Guidelines for the management of lower back pain refer to the importance of screening for CES. The primary treatment is prompt surgical decompression, ideally within the first 48 hours of diagnosis, which has been found to improve sensory and motor deficits. Performing delayed surgery on a patient with suspected CES will increase the likelihood of irreversible postoperative neurological deterioration. Furthermore, even if the patient is treated within the 48 hours limit, there is no concrete indication that all affected functions will be 100% recovered since CES outcomes are also determined by their symptoms at presentation.

There exist many causes of CES, but the most common cause is that of lumbar spine disc herniation and it occurs more frequently between the ages of 30-50. Other causes include prolapse, spinal neoplasms, hematomas, spinal abscesses, and significant injuries to the lower back. Typically, patients with suspected CES undergo a thorough neurological examination which includes the assessment of anal tone employing a Digital Rectal Examination or DRE. Multiple studies have been conducted to ascertain the accuracy of DRE for evaluating abnormal anal tone, but no definitive answer has been found. The divided opinions may be attributed to the extremely subjective nature of the examination and the lack of standardized methods. Generally, clinicians and doctors only recognize anal sphincter pressure changes 64% of the time, which is not precise enough to be used as one of the primary screening techniques for suspected CES before Magnetic Resonance Imaging (MRI). Therefore, the present invention features a rectal tonometer, a novel anal sphincter “tone” device to substitute DRE during suspected CES examinations.

Rectal tonometer devices currently implement water-filled catheters with multiple pressure ports. These prior devices measure the water pressure required to induce net positive flow and derive the rectal tone from this value. However, these devices lack the sensitivity to accurately identify rectal tone, and thus the “finger test” method is much more commonly used. While doctors are able to identify rectal tone from this test a majority of the time, it is still an inaccurate practice overall and does not provide any quantitative data since the finger test only results in a boolean value. Thus, there exists a present need for an accurate and sensitive rectal tonometer device that is able to determine rectal or vaginal tone based on quantitative data collected from the patient. The present invention features an air-filled sensor without the need for water filling and associated tubing, thus resulting in a more compact and less expensive device.

From previous studies, it is documented that anal sphincter pressure changes, when combined with history and examination findings suggestive of bladder, bowel, or sexual dysfunction, are an indicated sign to determine spinal pathology. Presented is an examination of the rectal tonometer's design specifications, performance, and key characteristics that make it stand out from the rest of the contemporary screening approaches. The present invention is intended for adult patients with suspected CES but can be adjusted for varying age ranges and different spinal cord injuries that affect the anorectal region (i.e. anal incontinence, anorectal malformations, reduced tone due to postoperative deterioration, central spinal conditions, among others) and require real-time feedback protocols.

### BRIEF SUMMARY OF THE INVENTION

It is an objective of the present invention to provide devices, methods, and systems that allow for quantitative measurement of a rectal tone, as specified in the independent claims. Embodiments of the invention are given in the dependent claims. Embodiments of the present invention can be freely combined if they are not mutually exclusive.

The present invention features a rectal tonometer device for quantitatively detecting and measuring a rectal tone of a patient. In some embodiments, the device may comprise a main body comprising a cavity within and a deformable tip, comprising a pressure sensor, partially disposed in the cavity and extending from the main body. Deforming the tip may actuate the pressure sensor. The rectal tonometer device may further comprise a circuit board disposed within the cavity, communicatively coupled to the deformable tip capable of accepting a pressure signal from the pressure sensor and measuring, based on the pressure signal, the rectal tone of the patient.

The present invention features a method for quantitatively detecting and measuring a rectal tone of a patient. In some embodiments, the method may comprise a rectal tonometer device comprising a main body comprising a cavity within and a deformable tip, comprising a pressure sensor, partially disposed in the cavity and extending from the main body. Deforming the tip may actuate the pressure sensor. The device may further comprise a circuit board disposed within the cavity. The method may further comprise placing a finger cot over the deformable tip, inserting the deformable tip into a sphincter region of the patient, accepting, by the pressure sensor, a pressure signal from the sphincter region of the patient, and measuring, based on the pressure signal, the rectal tone of the patient.

The present invention features a system for diagnosing a complication affecting an anorectal region of the patient, the system comprising a rectal tonometer device comprising a main body comprising a cavity within and a deformable tip, comprising a pressure sensor, partially disposed in the cavity and extending from the main body. Deforming the tip may actuate the pressure sensor. The device may further comprise a circuit board disposed within the cavity, communicatively coupled to the deformable tip. The circuit may be capable of accepting a pressure signal from the pressure sensor and transmitting the pressure signal through a communication component. The system may further comprise the communication component coupling the rectal tonometer device to a computing device. The system may further comprise the computing device comprising a processor capable of accepting the pressure signal from the rectal tonometer device by the communication component, generating, based on the pressure signal, a data log, and determining, based on the data log, a diagnosis of the complication.

One of the unique and inventive technical features of the present invention is the implementation of a deformable tip comprising a pressure sensor to measure a rectal tone of a patient. Without wishing to limit the invention to any theory or mechanism, it is believed that the technical feature of the present invention advantageously provides for efficient and accurate diagnosis of CES in a patient. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a rectal tonometer device for quantitatively detecting and measuring a rectal tone of a patient of the present invention, deconstructed into its individual components.

FIG. 2 shows a flow chart of a method for quantitatively detecting and measuring a rectal tone of a patient of the present invention.

FIGS. 3A-31 show a method of assembling the rectal tonometer device of the present invention.

FIG. 4 shows an alternate embodiment of the rectal tonometer device of the present invention with a display component.

FIG. 5 shows an embodiment of a data log retrieved by the rectal tonometer device of the present invention.

FIG. 6 shows a plurality of pressure signals gathered from testing the rectal tonometer device of the present invention at various PSI levels.

FIG. 7 shows a graph of pressure increases relative to time for a single test of the rectal tonometer device of the present invention.

FIG. 8 shows a graph of linear interpolation between the rectal tonometer device of the present invention and regulator pressures.

FIG. 9 shows a diagram of a saguaro design of the rectal tonometer of the present invention.

FIG. 10A shows a view of the deformable tip of the present invention in a neutral state.

FIG. 10B shows a view of the deformable tip of the present invention in a deformed state.

FIG. 11A shows a bottom view of the deformable tip of the present invention in a neutral state.

FIG. 11B shows a bottom view of the deformable tip of the present invention in a deformed state.

### DETAILED DESCRIPTION OF THE INVENTION

Following is a list of elements corresponding to a particular element referred to herein:
100 rectal tonometer device
110 main body
120 deformable tip
121 pressure sensor
130 circuit board
140 tip stopper
150 display component
200 communication component
300 computing device

The present invention features a rectal tonometer device (100) for quantitatively detecting and measuring a rectal tone of a patient, the device (100) comprising a main body (110) and a pressure-sensitive tip (120) extending from the main body (110). Deforming the tip (120) may generate a pressure signal. The device (100) may further comprise a processing device (130), communicatively coupled to the tip (120), capable of measuring, based on the pressure signal, the rectal tone of the patient. The processing device (130) may comprise a circuit board. The circuit board may comprise a microprocessor capable of executing computer-readable instructions and a memory component operatively coupled to the microprocessor, comprising a plurality of computer-readable instructions. The pressure-sensitive tip (120) may comprise a deformable tip comprising a pressure sensor (121).

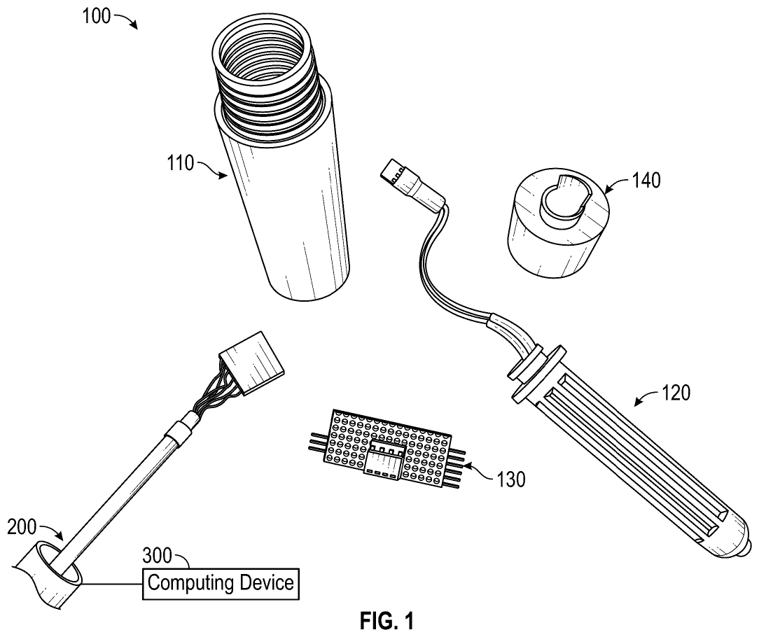

Referring now to FIG. 1, the present invention features a rectal tonometer device (100) for quantitatively detecting and measuring a rectal tone of a patient. In some embodiments, the device (100) may comprise a main body (110) comprising a cavity within and a deformable tip (120), comprising a pressure sensor (121), partially disposed in the cavity and extending from the main body (110). Deforming the tip (120) may actuate the pressure sensor (121). The rectal tonometer device (100) may further comprise a circuit board (130) disposed within the cavity, communicatively coupled to the deformable tip (120), comprising a microprocessor capable of executing computer-readable instructions and a memory component operatively coupled to the microprocessor, comprising a plurality of computer-readable instructions. The plurality of computer-readable instructions may comprise accepting a pressure signal from the pressure sensor (121) and measuring, based on the pressure signal, the rectal tone of the patient.

In some embodiments, the device (100) may further comprise a tip stopper (140) connecting the deformable tip (120) to the main body (110). The device (100) may be used to diagnose a complication affecting an anorectal region of the patient. The complication may be selected from a group comprising cauda equina syndrome, anal incontinence, anorectal malformations, reduced tone due to postoperative deterioration, central spinal conditions, and a combination thereof. In some embodiments, the device (100) may further comprise a display component (150) disposed on the main body (110) communicatively coupled to the circuit board (130). In such an embodiment, the memory component may further comprise computer-readable instructions for displaying the pressure signal on the display component (150) in units of mmHg. The display component (150) may comprise an LED display. In some embodiments, the device (100) may further comprise a fastening ring operatively coupled to a base of the deformable tip (120) for fastening a finger cot disposed over the deformable tip (120) in place.

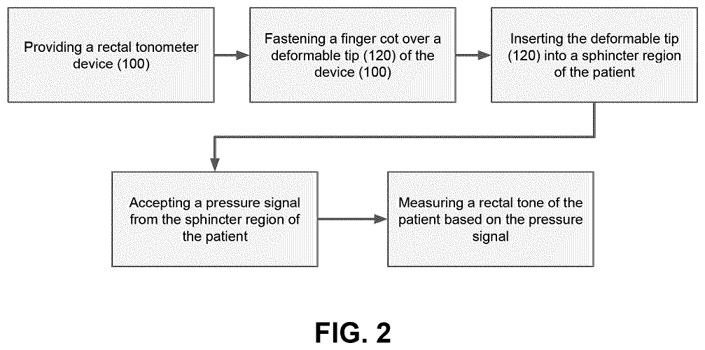

Referring now to FIG. 2, the present invention features a method for quantitatively detecting and measuring a rectal tone of a patient. In some embodiments, the method may comprise providing a rectal tonometer device (100) comprising a main body (110) comprising a cavity within and a deformable tip (120), comprising a pressure sensor (121), partially disposed in the cavity and extending from the main body (110). Deforming the tip (120) may actuate the pressure sensor (121). The device (100) may further comprise a fastening ring operatively coupled to a base of the deformable tip (120), and a circuit board (130) disposed within the cavity, communicatively coupled to the deformable tip (120), comprising a microprocessor capable of executing computer-readable instructions and a memory component comprising a plurality of computer-readable instructions. The method may further comprise fastening, by the fastening ring, a finger cot over the deformable tip (120), inserting the deformable tip (120) into a sphincter region of the patient, accepting, by the pressure sensor (121), a pressure signal from the sphincter region of the patient, and measuring, by the circuit board (130), based on the pressure signal, the rectal tone of the patient.

In some embodiments, the rectal tonometer device (100) may further comprise a tip stopper (140) connecting the deformable tip (120) to the main body (110). The present method may be used to diagnose a complication affecting an anorectal region of the patient. The complication may be selected from a group comprising cauda equina syndrome, anal incontinence, anorectal malformations, reduced tone due to postoperative deterioration, central spinal conditions, and a combination thereof. In some embodiments, the rectal tonometer device (100) may further comprise a display component (150) disposed on the main body (110) communicatively coupled to the circuit board (130). The memory component may further comprise computer-readable instructions for displaying the pressure signal on the display component (150) in units of mmHg. The display component (150) may comprise an LED display.

Referring now to FIG. 1, the present invention features a system for diagnosing a complication affecting an anorectal region of the patient, the system comprising a rectal tonometer device (100) comprising a main body (110) comprising a cavity within and a deformable tip (120), comprising a pressure sensor (121), partially disposed in the cavity and extending from the main body (110). Deforming the tip (120) may actuate the pressure sensor (121). The device (100) may further comprise a circuit board (130) disposed within the cavity, communicatively coupled to the deformable tip (120), comprising a microprocessor capable of executing computer-readable instructions and a memory component comprising a plurality of computer-readable instructions. The computer-readable instructions may comprise accepting a pressure signal from the pressure sensor (121), and transmitting the pressure signal through a communication component (200). The system may further comprise the communication component (200) coupling the rectal tonometer device (100) to a computing device (300). The system may further comprise the computing device (300) comprising a processor capable of executing computer-readable instructions and a memory component comprising computer-readable instructions. The computer-readable instructions may comprise accepting the pressure signal from the rectal tonometer device (100) by the communication component (200), generating, based on the pressure signal, a data log, and determining, based on the data log, a diagnosis of the complication.

In some embodiments, determining the diagnosis comprises determining whether the pressure signal falls above or below a predetermined threshold on the data log. The complication may be selected from a group comprising cauda equina syndrome, anal incontinence, anorectal malformations, reduced tone due to postoperative deterioration, central spinal conditions, and a combination thereof. The rectal tonometer device (100) may further comprise a fastening ring operatively coupled to a base of the deformable tip (120) for fastening a finger cot disposed over the deformable tip (120) in place.

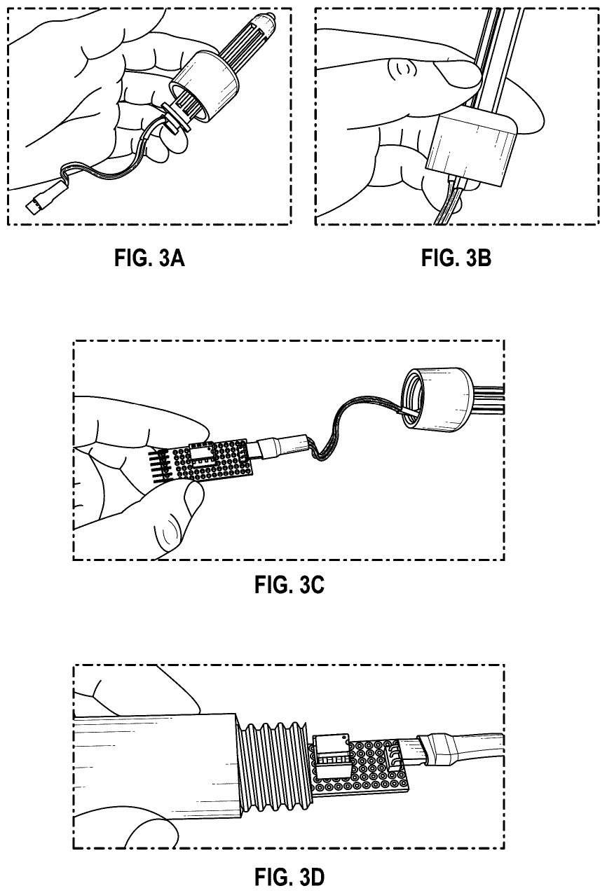

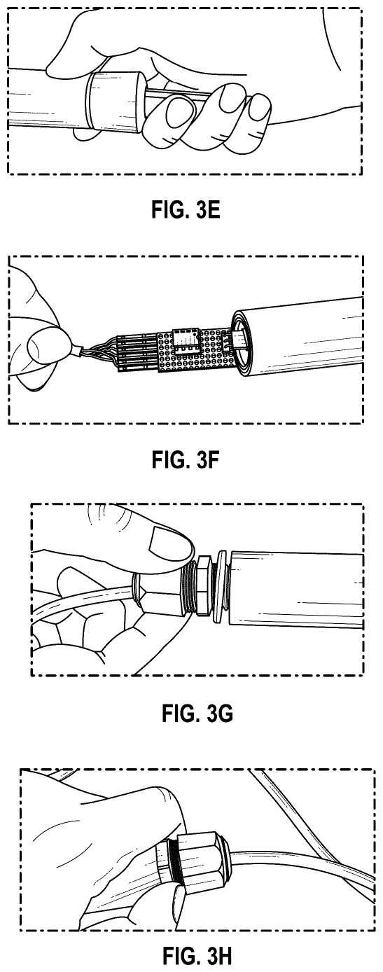

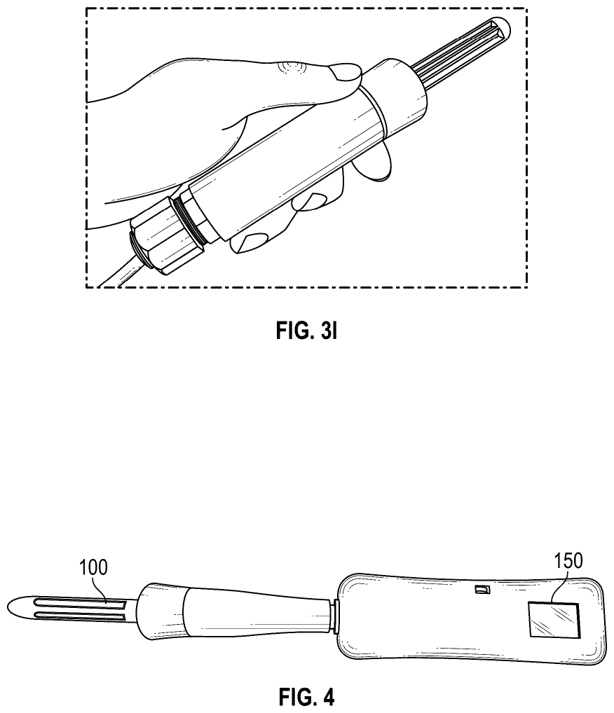

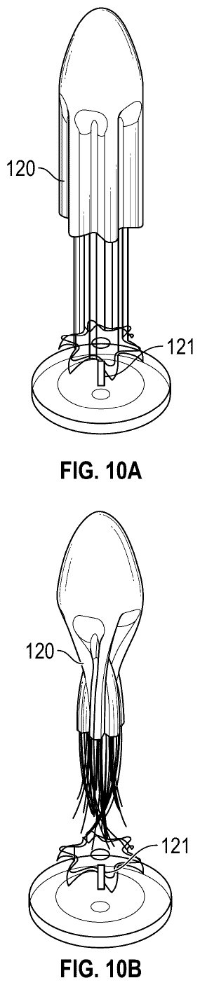

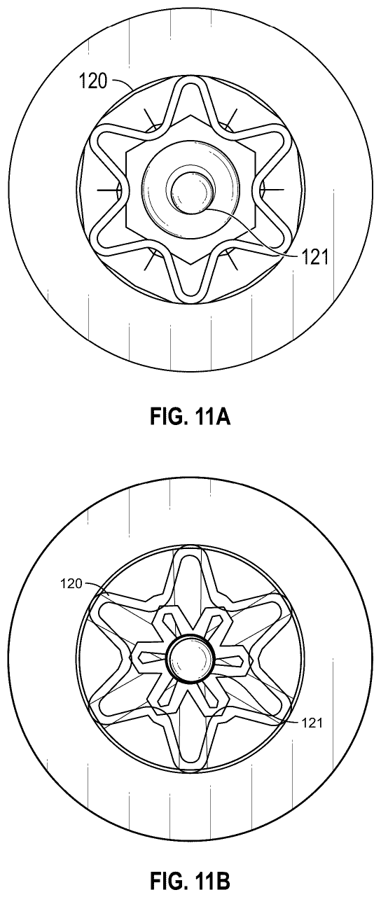

Referring now to FIGS. 3A-31, assembling the rectal tonometer device of the present invention may comprise gathering all necessary components, comprising a deformable tip with an attached pressure sensor, a microcontroller (MCU) circuit, an electronics housing, and a cable with a strain relief connector. Assembling the device may further comprise placing the tip on a plastic holder and sliding it until it stops moving and connecting the MCU circuit to the pressure sensor. Assembling the device may further comprise inserting the circuit into the main housing from the top, screwing the circuit in without twisting or turning the tip, pulling the circuit from the bottom of the main housing, and connecting the cable to the circuit. Assembling the device may further comprise pushing the circuit back into the housing, screwing the strain relief connector, and tightening the USB cable to the strain relief connector. Once the cable is connected, a finger cot should be placed on the deformable tip before inserting it into the patient's anorectal region.

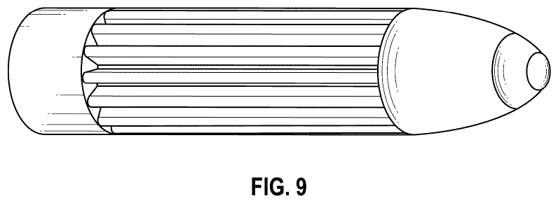

Referring now to FIG. 9, the present invention features a rectal tonometer tip with a “saguaro design.” In some embodiments, this design is ribbed, such that stiffness in an axial direction is greater than stiffness in a radial direction. This may enable insertion into a rectal region of a patient without the tip buckling, while at the same time offering sensitivity to rectal tone in the radial direction.

In some embodiments, the deformable tip may comprise a hollow body having a cylindrical shape. The cavity within the hollow body may be suitable for a pressure sensor to be removably disposed within the hollow body. The deformable tip may further comprise a plurality of ridges disposed radially around the hollow body, each ridge having a length equal to that of the hollow body. In some embodiments, the deformable tip has 5 to 7 ridges. In some embodiments, the ridges are equally distanced around the circumference of the hollow body. Applying pressure to the deformable tip may cause the ridges to deform and compress inwards to press on the pressure sensor, thus generating a pressure signal. This tip design allows for greater accuracy and sensitivity for the detection of rectal or vaginal tone when compared to prior designs.

### Example

The following is a non-limiting example of the present invention. It is to be understood that said example is not intended to limit the present invention in any way. Equivalents or substitutes are within the scope of the present invention.

The rectal tonometer was primarily made from a deformable rubber probe resistant to axial buckling and conformal to the patient's rectoanal geometry. It possessed a cylindrical shape with ridges spanning lengthwise to increase the compressibility of the material. This also allowed for a far more accurate measurement of pressure changes along the rectoanal canal. The following sections focus on the development of the rectal tonometer probe, circuitry design, and guided user interface creation.

The model's primary design and testing were accomplished with the use of a computer-aided design (CAD) package (SolidWorks Corporation, Concord, MA) which permitted rapid modifications to the element's properties and dimensions. The design fundamentally consisted of a single cylindrical element with six ridges around its circumference to increase elasticity in key locations and a hollow interior to improve circuitry connection and readings. It was termed the “Saguaro Model” due to its apparent similarity to the arborescent cactus species.

Dimensions were regular with the reviewed literature in adult rectal anatomy: an inner diameter of 14 mm and a length of 30 mm. Furthermore, Young's modulus used for the probe's material was 12 MPa and its Poisson's ratio was 0.48, which was consistent with SolidWorks' materials database corresponding to silicone-rubber compounds. Before the creation of a physical prototype, however, a compressibility and viability test under radial distributed loads was performed using the CAD package's finite element analysis (FEA) method. The test was intended to simulate anal sphincter behavior, particularly its peak pressures and total displacement. The model was then 3D printed to continue with the examinations.

Because of the varying range of anorectal tones found in the literature, 20 mmHg to 370 mmHg, a high-accuracy pressure sensor circuit for the rectal tonometer was developed. The electrical circuit involved the application of a high-temperature precision integrated silicon pressure sensor MPXV5050V series (NPX Semiconductors, Austin, TX) that fed through an 8-Pin Microcontroller (MCU) with 10-Bit ADC (Microchip Technology, Chandler, AZ) to convert the analog input into a digital signal. Afterward, the converted output is sent to a laptop/computer via a USB to TTL Serial Cable (FTDI Chip, Glasgow, UK) where the operator displays the pressure changes and conducts further analyses.

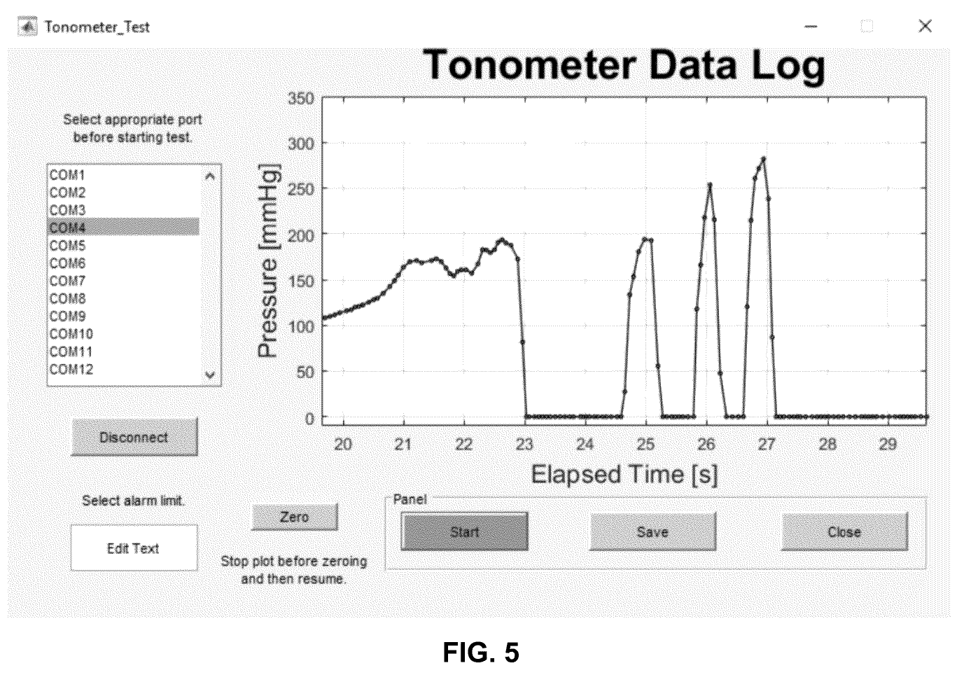

To improve the real-time data acquisition of the rectal tonometer and facilitate post-processing methods, a MATLAB (MathWorks, Natick, MA) Guided-User Interface (GUI) with a standalone downloadable application was developed to be used in combination with the medical device (FIG. 5).

The GUI comprised of a port selection mode, where the operator could select and connect from a list of available serial ports, an alarm limit box, which could be used to trigger an alarm whenever the anal tone was below or above a pre-defined threshold (units in millimeter of mercury), a zeroing button, used for calibration purposes, and a button panel for starting, stopping, or saving the data as well as closing the GUI. Once the data was saved, the information was retrieved in the form of a text file on the same document path the application was downloaded and initialized from.

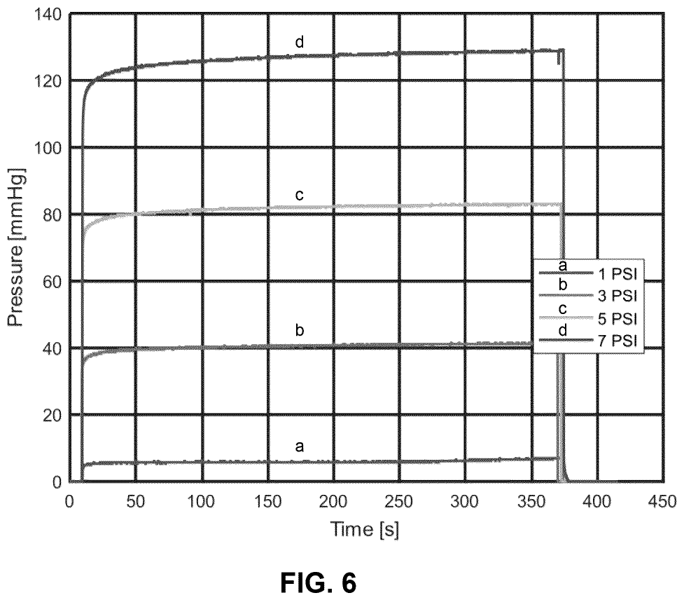

To examine the performance of the rectal tonometer device under varying radial pressures and its reliability over time, a calibration test was performed. The test utilized a calibration chamber made of schedule 40 PVC pipes with a maximum pressure rating of 450 PSI, which is about 23271.7 mmHg (FIG. 6). A hole approximately the size of the rubber probe was drilled into the base of the chamber to insert the rectal tonometer and an O-ring was used for sealing the aperture. Furthermore, the chamber was connected to a nitrogen gas tank through a hose barbed with a straight fitting style to provide the pressure, and a secondary regulator with a bleeder valve was used for decreasing the pressure range to an acceptable value for the rectal tonometer's pressure sensor specifications (maximum sensor pressure: 7.25 PSI) Toto keep the rubber probe in place during the calibration procedure and provide an air-tight connection, a chamber fitting was designed and then 3D printed. It had an octagonal opening to attach to the chamber base and a threaded interior to screw the rectal tonometer in place.

The preliminary pressure sensor calibration test consisted of applying increments of 2 PSI (103.43 mmHg) from the nitrogen gas tank to the rectal tonometer's rubber probe, recording the sensor's steady pressures, and saving them on a text file so that comparison with the regulator's readings was possible. The pressure range was from 1 to 7 PSI (51.71 to 362 mmHg). The secondary calibration procedure comprised a step-rate test model where the pressure applied to the probe was continuously increased by 1 PSI every 50 seconds until reaching the limit of 7 PSI. The pressure was increased manually by rotating the regulator approximately every 50 seconds.

The rectal tonometer's probe was capable of performing within the specified pressure range successfully and consistently. FIG. 6 illustrates the behavior of the pressure sensor for three different examinations on the same rectal tonometer. The lines represent the pressures selected/read on the nitrogen gas tank whereas the Y-axis represents the GUI-measured pressures. The maximum values per increment are as follows: 7.36 mmHg, 41.98 mmHg, 83.53 mmHg, and 129.55 mmHg, respectively.

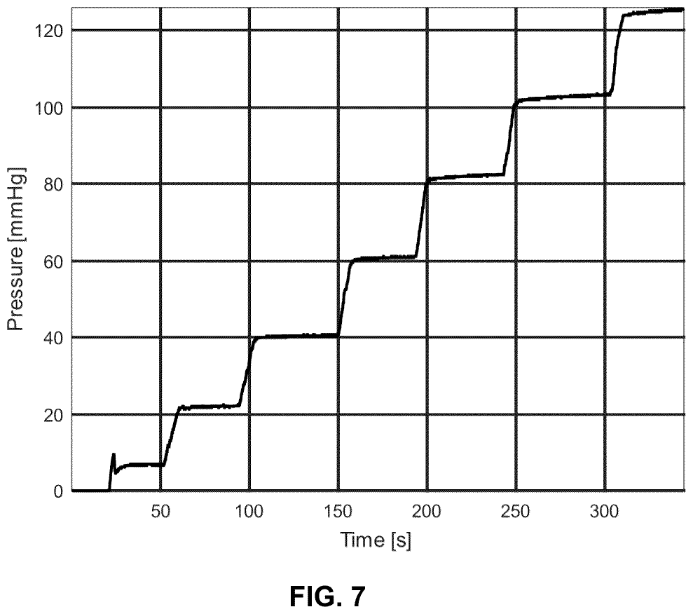

On the other hand, FIG. 7 exhibits the pressure increase relative to time for a single test. It is important to consider that oscillations due to untimed rotations were minimal and did not affect the results substantially.

As FIG. 6 demonstrates, the pressures increased rapidly at the start of the test and started to stabilize after approximately 20 seconds. However, as the regulator's pressure was incremented, it can be seen that it took more time for the sensor's values to become constant. This could be attributed to the time it takes the nitrogen gas to fill the volume of the calibration chamber and the pressure loads applied to the fittings. Once the chamber was filled, the rubber probe began to deform due to the compression of the gas, and this deformation allowed gas to leak from the chamber base.

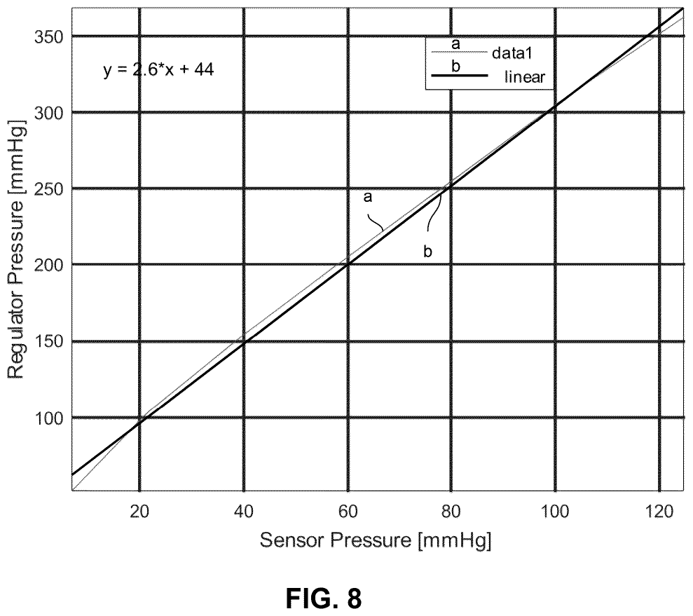

For the step-rate calibration test, the peaks showed consistency until arriving at about 80 mmHg where they started to diverge from their constant values (horizontal line). Taking the ratio of GUI-measured sensor pressure over the regulator's, it is found that the calibration equation was Y=2.6X+44. FIG. 8 illustrates the accuracy of the linear fit and the equation of the line. In conclusion, the rectal tonometer was capable of measuring the anorectal “tone” with an accuracy of +/−11.43 mmHg and time response of 15 milliseconds.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A rectal tonometer device (100) for quantitatively detecting and measuring a rectal tone of a patient, the device (100) comprising a main body (110), a pressure-sensitive tip (120) extending from the main body (110), configured to generate a pressure signal upon deformation, and a processing device (130) communicatively coupled to the tip (120), configured to measure, based on the pressure signal, the rectal tone of the patient.

2. The rectal tonometer device (100) of claim 1, wherein the processing device (130) comprises a circuit board.

3. The rectal tonometer device (100) of claim 2, wherein the circuit board comprises a microprocessor configured to execute computer-readable instructions and a memory component operatively coupled to the microprocessor, comprising a plurality of computer-readable instructions.

4. The rectal tonometer device (100) of claim 1, wherein the pressure-sensitive tip (120) comprises a deformable tip comprising a pressure sensor (121).

5. The rectal tonometer device (100) of claim 1 further comprising a tip stopper (140) connecting the tip (120) to the main body (110).

6. The rectal tonometer device (100) of claim 1 further comprising a display component (150) disposed on the main body (110), communicatively coupled to the processing device (130).

7. The rectal tonometer device (100) of claim 6, wherein the processing device (130) is further configured to display the pressure signal on the display component (150) in units of mmHg.

8. The rectal tonometer device (100) of claim 6, wherein the display component (150) comprises an LED display.

9. The rectal tonometer device (100) of claim 1 further comprising a fastening ring operatively coupled to a base of the tip (120), configured to fasten a finger cot disposed over the tip (120) in place.

10. The rectal tonometer device (100) of claim 1, wherein the tip (120) further comprises longitudinally aligned ribs such that stiffness in an axial direction is greater than stiffness in a radial direction.

11. A rectal tonometer device (100) for quantitatively detecting and measuring a rectal tone of a patient, the device (100) comprising:
a. a main body (110) comprising a cavity within;
b. a deformable tip (120), comprising a pressure sensor (121), partially disposed in the cavity and extending from the main body (110), configured to actuate the pressure sensor (121) when deformed; and
c. a circuit board (130) disposed within the cavity, communicatively coupled to the deformable tip (120), comprising a microprocessor configured to execute computer-readable instructions and a memory component operatively coupled to the circuit board (130), comprising a plurality of computer-readable instructions for:
i. accepting a pressure signal from the pressure sensor (121); and
ii. measuring, based on the pressure signal, the rectal tone of the patient.

12. The rectal tonometer device (100) of claim 11 further comprising a tip stopper (140) connecting the deformable tip (120) to the main body (110).

13. The rectal tonometer device (100) of claim 11 further comprising a display component (150) disposed on the main body (110), communicatively coupled to the circuit board (130).

14. The rectal tonometer device (100) of claim 13, wherein the memory component further comprises computer-readable instructions for displaying the pressure signal on the display component (150) in units of mmHg.

15. The rectal tonometer device (100) of claim 13, wherein the display component (150) comprises an LED display.

16. The rectal tonometer device (100) of claim 11 further comprising a fastening ring operatively coupled to a base of the deformable tip (120), configured to fasten a finger cot disposed over the deformable tip (120) in place.

17. The rectal tonometer device (100) of claim 11, wherein the tip (120) further comprises longitudinally aligned ribs such that stiffness in an axial direction is greater than stiffness in a radial direction.

18. A system for diagnosing a complication affecting an anorectal region of the patient, the system comprising:
a. a rectal tonometer device (100) comprising:
i. a main body (110) comprising a cavity within;
ii. a deformable tip (120), comprising a pressure sensor (121), partially disposed in the cavity and extending from the main body (110), configured to actuate the pressure sensor (121) when deformed; wherein the tip (120) further comprises longitudinally aligned ribs such that stiffness in an axial direction is greater than stiffness in a radial direction;
iii. a circuit board (130) disposed within the cavity, communicatively coupled to the deformable tip (120), comprising a microprocessor configured to execute computer-readable instructions and a memory component operatively coupled to the microprocessor, comprising a plurality of computer-readable instructions for:
A. accepting a pressure signal from the pressure sensor (121); and
B. transmitting the pressure signal through a communication component (200); and
iv. the communication component (200) coupling the rectal tonometer device (100) to a computing device (300); and
b. the computing device (300) comprising a processor configured to execute computer-readable instructions and a memory component operatively coupled to the processor, comprising computer-readable instructions for:
i. accepting the pressure signal from the rectal tonometer device (100) by the communication component (200);
ii. generating, based on the pressure signal, a data log; and
iii. determining, based on the data log, a diagnosis of the complication.

19. The system of claim 18, wherein determining the diagnosis comprises determining whether the pressure signal falls above or below a predetermined threshold on the data log.

20. The system of claim 18, wherein the rectal tonometer device (100) further comprises a fastening ring operatively coupled to a base of the deformable tip (120), configured to fasten a finger cot disposed over the deformable tip (120) in place.
